(19) United States
(12) Patent Application Publication
(10) Pub. No.: US 2025/0001054 A1
(43) Pub. Date: Jan. 2, 2025

(54) BONE JOINT MATERIAL, AND METHOD FOR PRODUCING BONE JOINT MATERIAL

(71) Applicant: GUNZE LIMITED, Ayabe-shi, Kyoto (JP)

(72) Inventors: Hidetoshi ARIMURA, Kyoto (JP); Mai TSUKAHARA, Kyoto (JP)

(21) Appl. No.: 18/708,480

(22) PCT Filed: Nov. 7, 2022
(86) PCT No.: PCT/JP2022/041420

§ 371 Date: May 8, 2024

(30) Foreign Application Priority Data
Nov. 10, 2021 (JP) 2021-183473

(51) Int. Cl. A61L 31/06; A61L 31/14

(52) U.S. Cl. CPC A61L 31/06; A61L 31/14; A61L 31/148; A61L 2430/02

(57) ABSTRACT

The present invention aims to provide an osteosynthesis material having high strength and an appropriate decomposition rate while being bendable by hand, and a method for producing the osteosynthesis material. Provided is an osteosynthesis material to be used for bone fixation at a fracture site, the osteosynthesis material consisting of poly-L-lactic acid, the osteosynthesis material having a bending resistance at 37° C. of 10 N or higher and a bending resistance at 70° C. of 5 N or lower.

### TECHNICAL FIELD

The present invention relates to an osteosynthesis material having high strength and an appropriate decomposition rate while being bendable by hand, and a method for producing the osteosynthesis material.

### BACKGROUND ART

Conventional osteosynthesis materials used for fixing bones until fractures heal include wires, plates, screws, pins, staples, clips, and rods that are made of stainless steel, ceramics, or the like. Metal or ceramic osteosynthesis materials, however, are not absorbed in the human body and thus require removal by reoperation after healing. Moreover, while these osteosynthesis materials have bending strength sufficient for practical use (e.g., around 323 N/mm² in the case of SUS-316 stainless steel osteosynthesis materials, around 245 to 490 N/mm² in the case of ceramic osteosynthesis materials), they are too rigid compared to human bone. These materials thus may scrape the bone in the area to which they are applied, or cause local osteolysis, decreased strength of new bone, or growth retardation of regenerated bone due to sustained stimulation.

To overcome the above issues, osteosynthesis materials made of bioabsorbable materials, such as poly-L-lactic acid, have been developed. For example, Patent Literature 1 discloses a biodegradable and resorbable molded article (osteosynthesis material) for surgery. The article is made of a polylactic acid molded article with a specific viscosity average molecular weight, a specific bending strength in compression, and a specific degree of crystallinity.

### CITATION LIST

### Patent Literature

Patent Literature 1: JP 2860663 B

### SUMMARY OF INVENTION

### Technical Problem

Such osteosynthesis materials made of poly-L-lactic acid have favorable characteristics that they can fix bones with sufficient strength until bone regeneration is achieved, and are eventually absorbed in the body. However, because of their high strength, conventional osteosynthesis materials made of poly-L-lactic acid require dedicated tools to reshape them according to the shape of the area to which they are applied. In response to this, osteosynthesis materials made of copolymers of poly-L-lactic acid with other bioabsorbable polymers or mixtures of poly-L-lactic acid with additives have been proposed. These osteosynthesis materials can become bendable by hand without any dedicated tool but only by being warmed in hot water. Unfortunately, while osteosynthesis materials made of copolymers containing poly-L-lactic acid or mixtures with additives are bendable by hand, they decompose faster in the body and may have difficulty in maintain sufficient strength until bone regeneration is achieved.

In view of the situation in the art, the present invention aims to provide an osteosynthesis material having high strength and an appropriate decomposition rate while being bendable by hand, and a method for producing the osteosynthesis material.

### Solution to Problem

The present invention relates to an osteosynthesis material to be used for bone fixation at a fracture site, the osteosynthesis material consisting of poly-L-lactic acid, the osteosynthesis material having a bending resistance at 37° C. of 10 N or higher and a bending resistance at 70° C. of 5 N or lower.

The present invention is described in detail below.

The osteosynthesis material of the present invention consists of poly-L-lactic acid.

The osteosynthesis material of the present invention consists of poly-L-lactic acid alone (homopolymer), not a copolymer or a mixture. The osteosynthesis material thus has a decomposition rate that allows bone fixation with sufficient strength until bone regeneration is achieved and quick absorption in the body after bone regeneration.

The poly-L-lactic acid preferably has a weight average molecular weight of 100000 or greater and 500000 or less.

A weight average molecular weight of not less than the lower limit can lead to higher strength. A weight average molecular weight of not greater than the upper limit allows quick absorption in the body after bone regeneration, reducing foreign body reaction in the body. The weight average molecular weight of the poly-L-lactic acid is preferably 100000 or greater, more preferably 150000 or greater and is preferably 500000 or less, more preferably 450000 or less. Here, the weight average molecular weight is a weight average molecular weight in terms of standard polystyrene determined by gel permeation chromatography (GPC). Specifically, the weight average molecular weight can be determined based on a polystyrene standard at a column temperature of 40° C. using chloroform as an eluent and an organic solvent-type column having a wide pore-size distribution.

The poly-L-lactic acid preferably has a degree of crystallinity of 10.0% or higher and 25.6% or lower. The poly-L-lactic acid having a degree of crystallinity within the above range makes it easy for the osteosynthesis material to satisfy the bending resistance at 37° C. and bending resistance at 70° C. described later. The degree of crystallinity of the poly-L-lactic acid is more preferably 13.4% or higher, still more preferably 15.4% or higher and is more preferably 23.3% or lower, still more preferably 22.0% or lower. The degree of crystallinity of the poly-L-lactic acid can be adjusted by adjusting the temperature or the time in heat treatment after stretching.

The degree of crystallinity can be measured by a conventionally known method, for example an X-ray diffractometry or differential scanning calorimetry (DSC). DSC is preferable for its simplicity. With DSC, the degree of crystallinity can be calculated by the formula below using the obtained heat of fusion of the sample.

$$Degree of crystallinity ( \% ) = ( Δ H / Δ H 0 ) \times 100$$

ΔH (J/g): heat of fusion of sample
ΔH0 (J/g): theoretical heat of fusion of perfect crystal of poly-L-lactic acid

The osteosynthesis material of the present invention has a bending resistance at 37° C. of 10 N or higher.

The osteosynthesis material having a bending resistance at 37° C. within the above range can have sufficient strength to fix a bone until bone regeneration is achieved.

The bending resistance at 37° C. can be adjusted by adjusting the weight average molecular weight or the degree of stretching of the poly-L-lactic acid.

The bending resistance at 37° C. can be measured by the following method.

FIG. 1 is a schematic view illustrating the method for measuring the bending resistance. As shown in FIG. 1, a jig 2 provided with two parallel protrusions, spaced from each other by 15 mm, is placed in a water bath 1 filled with water, with the protrusions facing up. Subsequently, an osteosynthesis material 3 is placed like a bridge over the two protrusions. At this time, the osteosynthesis material 3 is placed such that it is orthogonal to the extending direction of the protrusions and that the center of the osteosynthesis material 3 is positioned on the median line between the protrusions. Then, the water bath 1 is set to a water temperature of 37° C., and after the water reaches 37° C., the osteosynthesis material 3 is left to stand for two minutes to equalize the temperature of the entire osteosynthesis material 3 to the water temperature. Thereafter, an autograph (e.g., AGS-5KNX, available from Shimadzu Corporation) is used to apply a force to the center of the osteosynthesis material 3 at 2.0 mm/min and measure the bending resistance.

The bending resistance at 37° C. is preferably 10 N or higher, more preferably 12 N or higher. The upper limit of the bending resistance at 37° C. is not limited but is preferably 35 N or lower.

The osteosynthesis material of the present invention has a bending resistance at 70° C. of 5 N or lower.

The osteosynthesis material of the present invention has a bending resistance at 37° C. of 10 N or higher and a bending resistance at 70° C. of 5 N or lower, so that it can have high strength at around body temperature but become soft when immersed in hot water. The osteosynthesis material is thus deformable by hand and have high handleability in surgery. While being bendable by hand, the osteosynthesis material of the present invention, made of poly-L-lactic acid alone (homopolymer), does not decompose faster in the body unlike a copolymer or a mixture, and can fix a bone with sufficient strength until bone regeneration is achieved.

The bending resistance at 70° C. can be measured by the same method as the bending resistance at 37° C. except that the water temperature is set to 70° C.

The bending resistance at 70° C. can be adjusted by adjusting the degree of stretching of poly-L-lactic acid.

The bending resistance at 70° C. is preferably 5 N or lower, more preferably 3 N or lower. The lower limit of the bending resistance at 70° C. is not limited but is preferably 0.1 N or higher from the standpoint of the ease of forming into a desired shape.

The osteosynthesis material of the present invention may have any shape. It may have a shape of any conventional osteosynthesis material, such as a dumbbell shape or a plate shape.

The method for producing the osteosynthesis material of the present invention is not limited as long as the bending resistance at 37° C. and the bending resistance at 70° C. are satisfied. A preferred method includes stretching a plate consisting of poly-L-lactic acid in a specific range and then forming the plate into a shape of the osteosynthesis material.

The present invention also encompasses a method for producing the osteosynthesis material of the present invention, the method including forming a plate consisting of poly-L-lactic acid, stretching the plate by a factor of 1.1 or greater and 2.0 or less, and forming the stretched plate into a shape of the osteosynthesis material.

With the method for producing the osteosynthesis material of the present invention, first, a plate consisting of poly-L-lactic acid is formed.

The plate consisting of poly-L-lactic acid is formed by any method. For example, the plate may be formed by a conventionally known method such as injection molding, extrusion molding, or blow molding.

With the method for producing the osteosynthesis material of the present invention, next, the plate is stretched by a factor of 1.1 or greater and 2.0 or less.

A conventional osteosynthesis material made of poly-L-lactic acid is produced by stretching a plate by a factor of 4 or greater to improve the strength. With the method for producing the osteosynthesis material of the present invention, the plate consisting of poly-L-lactic acid obtained in the above step is stretched in the above range. This makes it easy for the osteosynthesis material to satisfy the bending resistance at 37° C. and the bending resistance at 70° C.

The stretching factor is preferably 1.1 or greater and preferably 1.5 or less. The stretching method is not limited but is preferably rolling, from the standpoint of production efficiency.

With the method for producing the osteosynthesis material of the present invention, heat treatment is preferably performed after the stretching.

Heat treatment on the stretched poly-L-lactic acid allows the degree of crystallinity of the poly-L-lactic acid to be in a more optimal range. The heat treatment is appropriately adjusted based on the temperature or the time. For example, the heat treatment is performed at a temperature of 70° C. or higher and 80° C. or lower for 45 minutes or longer and 120 minutes or shorter.

With the method for producing the osteosynthesis material of the present invention, subsequently, the stretched plate is formed into a shape of the osteosynthesis material.

The stretched plate consisting of poly-L-lactic acid is formed into the shape of the osteosynthesis material to give the osteosynthesis material. The plate may be formed into the shape of the osteosynthesis material by any conventionally known method such as cutting or punching.

The osteosynthesis material of the present invention is used for bone fixation at a fracture site. The osteosynthesis material of the present invention has very high handleability because it becomes deformable by hand when immersed in hot water, while maintaining the favorable characteristics of poly-L-lactic acid, i.e., sufficient strength and an appropriate decomposition rate.

### Advantageous Effects of Invention

The present invention can provide an osteosynthesis material having high strength and an appropriate decomposition rate while being bendable by hand, and a method for producing the osteosynthesis material.

### BRIEF DESCRIPTION OF DRAWINGS

FIG. 1 is a schematic view illustrating a method for measuring bending resistance.

FIG. 2 is a graph showing bending resistance evaluation results.

FIG. 3 is a graph showing decomposability evaluation results.

### DESCRIPTION OF EMBODIMENTS

In the following, the present invention is described in more detail with reference to examples. The present invention is not limited only to these examples.

### Example 1

### (1) Production of Osteosynthesis Material

Molten poly-L-lactic acid homopolymer (weight average molecular weight: 300000) was injection-molded into a plate (90 mm×90 mm×2 mm). The obtained plate was stretched by a factor of 1.1 using a rolling machine, heat-treated, and then cut into an osteosynthesis material with a total length of 23 mm, a thickness of 1.5 mm, and a width of 5.7 mm.

### (2) Measurement of Degree of Crystallinity

A measurement sample (10 mg) was cut out of the obtained osteosynthesis material. Subsequently, the measurement sample was sealed in a dedicated container, and subjected to differential scanning calorimetry using a differential scanning calorimeter (DSC-60, available from Shimadzu Corporation) at a temperature increase rate of 10° C./min in the range from 30° C. to 220° C. From the endotherm and exotherm obtained by the differential scanning calorimetry and the theoretical heat of fusion of a perfect crystal of polylactic acid, the degree of crystallinity was calculated. The degree of crystallinity was 14.4%.

### Comparative Example 1

Molten poly-L-lactic acid homopolymer (weight average molecular weight: 300000) was injection-molded into a plate (45 mm×45 mm×11.5 mm). The obtained plate was stretched by a factor of 6.0 using a rolling machine. The plate was then cut into an osteosynthesis material with a total length of 22 mm, a thickness of 1.5 mm, and a width of 5.1 mm.

### Comparative Example 2

An osteosynthesis material was obtained as in Comparative Example 1 except that the thickness after cutting was 1.0 mm.

### Comparative Example 3

LactoSorb 915-2110 (copolymer of L-lactic acid and glycolic acid, total length: 21.2 mm, width: 6.9 mm, thickness: 1.4 mm, available from Medical U & A Inc.) was used as an osteosynthesis material directly.

### Comparative Example 4

An osteosynthesis material was obtained as in Comparative Example 1 except that the thickness after cutting was 0.95 mm and the width after cutting was 5.5 mm.

### <Evaluation>

The osteosynthesis materials obtained in the examples and the comparative examples were evaluated by the following methods.

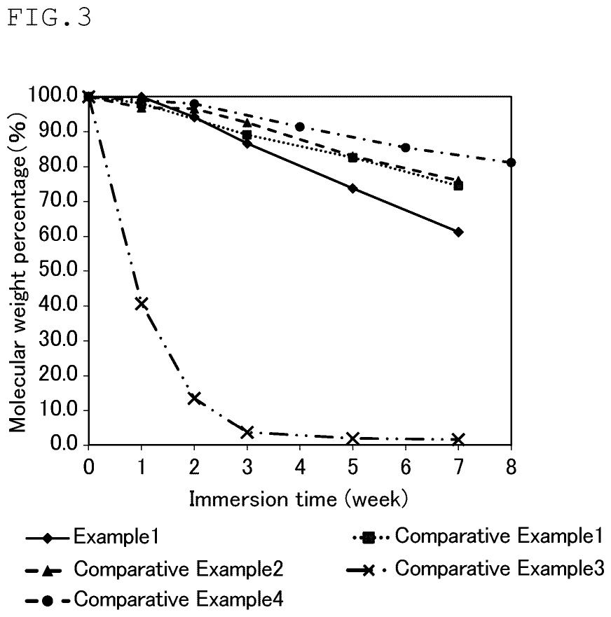

FIG. 2 and FIG. 3 show the results.

### (1) Evaluation of Bending Resistance

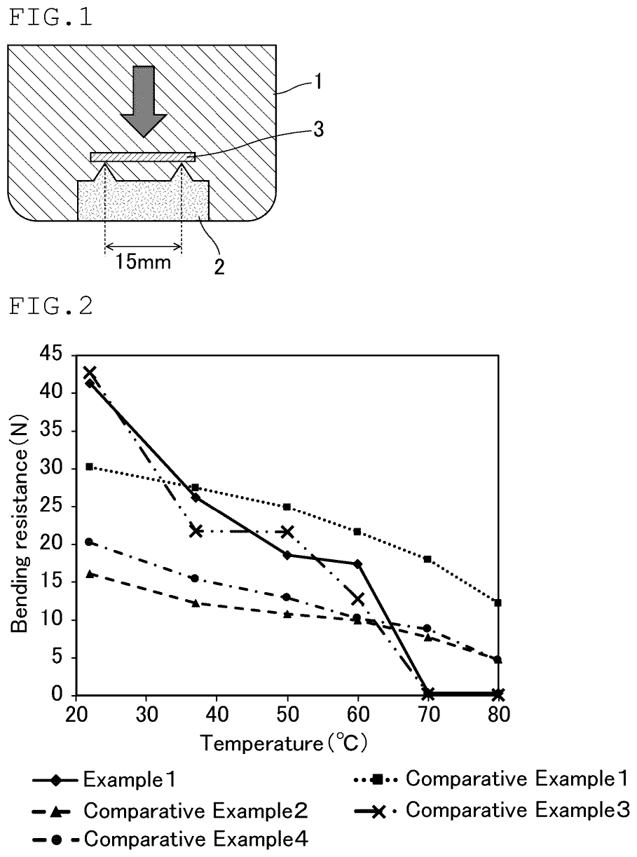

As shown in FIG. 1, a jig provided with two parallel protrusions, spaced from each other by 15 mm, was placed in a water bath filled with water, with the protrusions facing up. Subsequently, the osteosynthesis material was placed like a bridge over the two protrusions. At this time, the osteosynthesis material was placed such that it was orthogonal to the extending direction of the protrusions and that the center of the osteosynthesis material was positioned on the median line between the protrusions. Then, the water bath was set to a water temperature of 37° C., and after the water reached 37° C., the osteosynthesis material was left to stand for two minutes to equalize the temperature of the entire osteosynthesis material to the water temperature. Thereafter, an autograph (AGS-5KNX, available from Shimadzu Corporation) was used to apply a force to the center of the osteosynthesis material at 2.0 mm/min and measure the bending resistance at 37° C.

Subsequently, the same procedure was performed except that the water temperature was set to 50° C., 60° C., 70° C., or 80° C., and the bending resistance at the respective temperature was measured. Separately, the bending resistance at 22° C. was measured by the same procedure as above except that the measurement was performed at room temperature without the water bath. Table 1 and FIG. 2 show the results.

TABLE 1

| | | Temperature (° C.) | | | | | |
|---|---|---|---|---|---|---|---|
| | | 22 | 37 | 50 | 60 | 70 | 80 |
| Bending | Example 1 | 41.3 | 26.2 | 18.5 | 17.4 | 0.3 | 0.4 |
| resistance | Comparative | 30.3 | 27.6 | 24.9 | 21.7 | 18.0 | 12.2 |
| (N) | Example 1 | | | | | | |
| | Comparative | 16.1 | 12.2 | 10.8 | 10.0 | 7.7 | 4.8 |
| | Example 2 | | | | | | |
| | Comparative | 42.7 | 21.8 | 21.6 | 12.8 | 0.2 | 0.1 |
| | Example 3 | | | | | | |
| | Comparative | 20.2 | 15.4 | 12.9 | 10.3 | 8.8 | 4.8 |
| | Example 4 | | | | | | |

### (2) Evaluation of Decomposability

The osteosynthesis material was immersed in phosphate buffered saline (PBS) at a liquor ratio of 100 at 50° C., and the weight average molecular weight was measured by GPC after one week, two weeks, three weeks, five weeks, and seven weeks. With the molecular weight before immersion as 100%, the percentage of the weight average molecular weight (molecular weight percentage) was calculated, whereby the decomposability of the osteosynthesis material was evaluated. FIG. 3 shows the results. In Comparative Example 4, the measurement was performed after two weeks, four weeks, six weeks, and eight weeks.

### INDUSTRIAL APPLICABILITY

The present invention can provide an osteosynthesis material having high strength and an appropriate decomposition rate while being bendable by hand, and a method for producing the osteosynthesis material.

### REFERENCE SIGNS LIST

1 water bath
2 jig
3 osteosynthesis material

1. An osteosynthesis material to be used for bone fixation at a fracture site, the osteosynthesis material consisting of poly-L-lactic acid,
the osteosynthesis material having a bending resistance at 37° C. of 10 N or higher and a bending resistance at 70° C. of 5 N or lower.

2. A method for producing the osteosynthesis material according to claim 1, the method comprising:
forming a plate consisting of poly-L-lactic acid;
stretching the plate by a factor of 1.1 or greater and 2.0 or less; and
forming the stretched plate into a shape of the osteosynthesis material.
